(19) United States
(12) Patent Application Publication
(10) Pub. No.: US 2004/0039031 A1
(43) Pub. Date: Feb. 26, 2004

(54) Novel pharmaceutical compositions having an antidiabetic action, and process for their preparation

(76) Inventors: Nathalie Cugnardey, Lyon (FR); Nicolas Wiernsperger, Orlienas (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/432,386

(22) PCT Filed: Oct. 29, 2001
(86) PCT No.: PCT/EP01/12492

(30) Foreign Application Priority Data
Nov. 22, 2000 (FR) 0015066

(51) Int. Cl. A61K 31/44; A61K 31/403; A61K 31/175

(52) U.S. Cl. 514/354; 514/412; 514/592

(57) ABSTRACT

The present invention relates to the field of therapeutic chemistry and more particularly to the preparation of novel medicinal products for treating diabetes. Specifically, a subject of the invention is novel antidiabetic medicinal products consisting of a combination of two active principles that are effective via the oral route, at subliminal doses, formed from an antidiabetic biguanide and an antidiabetic sulfonamide, in combination or as a mixture with one or more inert, pharmaceutically acceptable excipients. The invention also relates to a process for the preparation of such medicinal products formed from a biguanide and a sulfonamide.

The present invention relates to the field of therapeutic chemistry and more particularly to novel medicinal products for treating diabetes.

More particularly, a subject of the invention is novel antidiabetic medicinal products formed from two known active principles whose effects are potentiated.

Specifically, a subject of the invention is novel antidiabetic medicinal products consisting of a combination of two active principles that are effective via the oral route, at subliminal dose, formed from an antidiabetic biguanide and an antidiabetic sulfonamide, in combination or as a mixture with one or more inert, pharmaceutically acceptable excipients.

More specifically, the antidiabetic of the biguanide type is chosen from metformin, buformin and phenformin, or a salt thereof with a therapeutically compatible mineral acid or organic acid.

The antidiabetic of the sulfonamide type corresponds to the general formula:

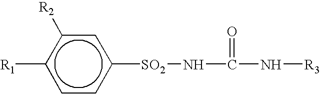

in which R₁ represents NH₂, CH₃, Cl, a group:

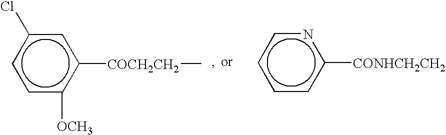

or alternatively R₁ forms with R₂ a group:

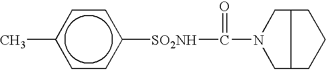

R₂ represents H or NH₂

R₃ represents a 4-n-butyl group, a cyclohexyl group, a group

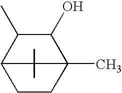

The main starting material of these constituents is that formed by glibenclamide:

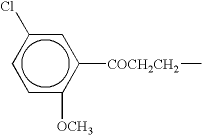

or by gliclazide:

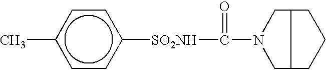

This combination of has quite exceptional qualities has been found that the combined administration of an antidiabetic agent of the biguanide type and of an antidiabetic agent of the sulfonamide type at low doses, at which they are inactive, results in a substantial reduction in glycaemia.

In particular, in rats made diabetic by destruction of the islets of Langerhans, by administration of streptozotocin, low doses of biguanide (for example metformin) and of sulfamide (for example glibenclamide) significantly improve the diabetes.

Patent EP 0 974 356 A1 in the name of LIPHA, which describes an embodiment of metformin and glibenclamide tablets in which it is ensured that the particle size of the glibenclamide grains is such that the bioavailability is guaranteed, was already known in the literature. This is the case described in the said document, in which not more than ten percent of the particles have a particle size of less than 2 μm, and not more than ten percent of the particles have a particle size of greater than 60 μm.

In addition, to prepare tablets, a binder is added to the powder mixture, in a proportion of from 2% to 4%. Working in this way has the serious drawback of ensuring such a relatively narrow particle size range, which requires specific means in order to be produced.

International patent WO 97/17975 (Gentili S.p.A.) also describes a combination of glibenclamide and metformin whose weight ratios are 1:100, and in particular 5 mg of glibenclamide and 500 mg of metformin. This dosage allows 15 mg of glibenclamide and 1500 mg of metformin to be administered three times a day.

The objective pursued is substantially different from the object of the present patent application.

Document WO 99/29314 (Bristol Myers Squibb Company) relates to a mixture of metformin in the form of a dibasic acid salt (fumarate or succinate) used separately or in combination with another antidiabetic agent. In this case, the antidiabetic agent described is glyburide. This preparation shows better storage due to its lower hygroscopicity and better flow of the powder. This preparation is also characterised by a markedly improved taste due to its lower solubility in water. This combination of metformin salts with a sulfamide or a sulfonylurea (glyburide [or glibenclamide], glimepiride, glipizide, gliclazide or chlorpropamide, etc.) acts on the ATP-dependent channel of β cells. The weight ratio of the metformin salt to the sulfonylurea can vary within the range from 300:1 to about 50:1. Thus, the examples in the said document describe a combination of 600 mg of metformin fumarate (2:1) and 5.0 mg of glyburide or glipizide.

No technical data relating to the preparation of such pharmaceutical formulations are given, with the exception of storage in the presence of humidity. No information is given regarding the antidiabetic activity of such a preparation.

The compositions according to the invention produce different effects.

It has been possible previously to determine that the daily dose of 0.5 mg/kg of glibenclamide is found to be inactive.

At higher doses that are already active (1 mg/kg/day) of sulfonamide (or 2 mg/kg/day), the administration of sulfonamide does not modify the glycaemia, but instead results in a significant increase in insulinaemia and a reduction in the level of circulating lactates.

It has been possible to determine that the minimum active dose of sulfonamide—for instance glibenclamide—is 2 mg/kg/day. Similarly, the minimum active dose of biguanide—for like-wise metformin—is a high dose at least equal to 30 mg/kg/day. This dose brings about a slight decrease in glycaemia, a return to normal of the triglycerides, a significant decrease in lactates and an increase in insulinaemia

In the abundant literature concerning the antihyperglycaemiant activity of metformin, the vast majority of the animal models used give active doses of between 100 and 200 mg/kg. These active doses in animals correspond to the confirmed therapeutic use in man, for equilibrating a diabetes, of a dosage ranging from 500 mg to 3 g per day depending on the severity of the diabetes.

The invention consists in that, by using an adapted animal model, it is indicated and demonstrated that a dosage of less than 500 mg per day for metformin may be used to treat a diabetic condition with reduced risks as regards compliance and tolerance.

It has also been shown that, at subliminal doses, synergy is observed between the effects of biguanide with sulfonamide. The combination of these two types of substance significantly decreases cholesterol and triglycerides, the level of which is very much increased in the case of rats treated with streptozotocin.

In addition, sulfonamide not only decreases lactates, but also inhibits the effects of biguanide, which has a tendency to increase them. The sum of the decreases in glycaemia obtained with the products administered separately (20 mg/kg/day) is substantially less than that obtained with the combination according to the invention.

| Product | Dose | Decrease in glycaemia | SE |
|---|---|---|---|
| Metformin | 30 mg/kg/day | 86.03 | ±4.29 |
| Glibenclamide | 2 mg/kg/day | 93.49 | ±1.15 |
| Combination | | 78.46 | ±0.42 |

** p > 0.01

There is thus, at these active doses, a potentiation of the effects of the two oral antidiabetics when they are administered in combination:

synergistic effect on the decrease in glycaemia,

potentiating effect on the decrease in the content of glycerol and triglycerides,

an increase in insulinaemia specific to sulfonamide,

a decrease in lactates, this effect being specific to glibenclamide, with suppression of the effects specific to the biguanides.

The combined use of these two types of molecule is rational when non-insulin-dependent diabetics (NIDDs) exhibit both hyperglycaemia and low insulinaemia.

In addition, in the case of hyperinsulinaemia, contrary to what might be thought, a disappearance of the hypersecretion of insulin is observed in the course of the development of the diabetes and the combination according to the invention becomes necessary.

In the compositions according to the invention, the biguanide/sulfonamide dose ratio ranges from 10:1 to 45:1, and by weight from 50:5 mg to 100:2.5 mg. This ratio thus differs substantially from that described in international patent WO 97/17975 (ratio 100:1) and in international patent WO 99/29814 (Bristol Myers).

This ratio is thus particularly advantageous since it allows a large decrease in the doses of biguanide to be envisaged.

The compositions according to the invention are in one of the forms suitable for oral administration, such as tablets, film-coated tablets, coated tablets, sugar-coated tablets, gel capsules, wafer capsules, pills, troches, lozenges, tablets splittable into small bars, granules, microgranules, microspheres and similar preparations.

To prepare these pharmaceutical forms, the biguanide and the sulfonamide are mixed with one or more inert, non-toxic, solid or liquid pharmaceutical excipients. Mention will be made in this respect of mineral fillers such as calcium carbonate, magnesium carbonate, tricalcium phosphate, magnesium phosphate, kaolin, talc, magnesium stearate, silicon dioxide, titanium dioxide, zirconium dioxide or colloidal silica. Organic fillers which may be mentioned are cellulose and its derivatives, alginates, carrageenates, chitosan derivatives, plant gums, for instance gum tragacanth, guar gum and its derivatives, xanthan gum, starches, maltodextrins and plant oils.

Pharmaceutical compositions containing the biguanide-sulfonamide antidiabetic combination are prepared by the current processes known to those skilled in the art. They will be understood more clearly on the basis of a detailed preparation example.

According to one particular characteristic of the compositions according to the invention, one formulation which is particularly advantageous is that corresponding to 30 mg/kg of metformin hydrochloride and 2 mg/kg of glibenclamide.

As described hereinabove, the doses of sulfonamide may be reduced by means of the compositions according to the invention. The doses of biguanide may be varied within wide proportions without this being an inconvenience for the manufacture.

The examples which follow illustrate the invention without, however, limiting it.

Pharmacological Study of the Biguanide-Sulfonamide Combination According to the Invention

Animals

40 male Wistar rats (Charles River, Saint Aubin les Elbeuf, France), with an average weight of 280 g, are used in this experiment. The animals are housed for one week in a standardised animal house, the following parameters of which are controlled:

day/night rhythm 7:00/19:00

temperature 22+/−1° C.;

hygrometry 50+-10%.

The animals have access, ad libitum, to drinking water and to a standard feed UAR A03.

Experiment

The forty rats are made diabetic by IP administration of 50 mg/kg of streptozotocin dissolved in physiological saline (a single administration). A non-insulin-dependent diabetes (NIDD) develops within two weeks and remains stable for at least one month.

The animals are selected 21 days after the administration of STZ, as a function of the value of the glycaemia of between 10 and 15 mM, which corresponds to an NIDD diabetes, and of their insulinaemia (value of between 15 and 20 μU/l). About 30% of the rats have a higher glycaemia with a low insulinaemia (IDD), associated with appreciable weight loss. These IDDs are removed from this experiment. 24 rats remain (two groups of 12 distributed randomly).

21 days after the administration of STZ, the selected rats are then treated either with glibenclamide or with metformin, or with the glibenclamide-metformin combination according to the invention.

1st Part of the Experiment

Two batches of six rats taken at random:

1 batch receives 1 mg/kg/day of glibenclamide orally in two doses, for eight days.

1 batch receives 2 mg/kg/day of glibenclamide orally in two doses, for eight days.

2nd Part of the Experiment

The other two batches drawn at random are then treated:

1 batch receives 30 mg/kg/day of metformin (in hydrochloride form) orally in two doses, for eight days. 30 mg/kg/day is the minimum active dose on this model and under the experimental conditions defined.

1 batch receives the combination metformin 30 mg/kg/day and glibenclamide 2 mg/kg/day orally (minimum active dose, chosen as a function of the results of the 1st part) in two doses, daily for eight days.

Biological Parameters Measured

For each rat, 100 μl of blood are collected by venepuncture in a caudal vein, onto heparin, at time D0 (before the administration of STZ), at time D21 (21 days after STZ, before the treatments) and at D29 (after eight days of treatment). The samples are immediately centrifuged (10 minutes at 4000 rpm) and the plasma is separated from the blood cells. The samples are frozen until the time of determination of the biological parameters.

Glycaemia, Cholesterol, Triglycerides, Lactic Acid

After thawing the plasma samples, the above parameters are determined by enzymatic methods, using a COBAS automated machine (Roche).

The glycaemia is measured by the hexokinase method; the cholesterol by the enzymatic final Randox point method; the triglycerides by the GPO-PAP method; the lactic acid by the lactic dehydrogenase method.

Insulinaemia

The circulating insulin is measured by radioimmunoassay using CEA kits.

The homogeny between human insulin and rat insulin is very large and the results obtained are at 95% of their true value.

All the methods are validated and systematically controlled with standards.

Statistical Analysis

The averages of the individual results obtained are affected by the error to be expected on the mean.

At D0 and D21, after an analysis of variance ANOVA, the absence of intergroup significance is analysed by a Student t test.

The efficacy of the treatments is evaluated between D29 and D21 by a t test adapted to paired series (for each rat, the value at D21 relative to D29 serves as the control).

Results

1/Determination of the Minimum Active Dose of Glibenclamide

In a study also carried out on NIDD rats, of STZ type, a daily dose of 0.5 mg/kg of glibenclamide was found to be inactive. Two stronger doses were thus tested, i.e. 1 mg/kg/day or 2 mg/kg/day.

The results regarding this test have been collated. At D0 before any treatment, the two batches of rats are comparable. Similarly, at D21, after the administration of streptozotocin, there is no difference between the two batches of animals. The administration of the low dose of glibenclamide does not modify the glycaemia. However, a significant increase in the insulinaemia and a decrease in the level of circulating lactates are observed.

The higher dose of glibenclamide significantly decreases the value of the glycaemia.

This significant decrease is observed with the Student t test for a paired series (pairs method). With this dosage, the lactate levels remain significantly reduced, whereas the insulinaemia is still higher on average (Table 3).

These results show that the dose of 2 mg/kg/day of glibenclamide constitutes the minimum active dose.

Under these conditions, the efficacy of this dose of glibenclamide was compared with that of metformin (30 mg/kg/day) and with the combination of these two minimum doses.

The results are collated in Table II.

TABLE I
Effects of glibenclamide administered for eight days at daily doses of 1 or 2 mg/kg/day
divided into two doses, on the biochemical parameters of diabetic rats

| | | | | | |
|---|---|---|---|---|---|
| Weight | Glycaemia | Cholesterol | Triglycerides | Lactates | Insulinaemia |
| (g) | mM/l | mM/l | mM/l | mM/l | μU/ml |

1 mg/kg/day

| | | | | | | |
|---|---|---|---|---|---|---|
| D0 | 291 | 6.39 +/− 0.17 | 1.03 +/− 0.04 | 0.87 +/− 0.05 | 2.19 +/− 0.07 | 12.8 +/− 0.8 |
| D21 | 253 | 14.22 +/− 0.65 | 1.65 +/− 0.08 | 1.04 +/− 0.10 | 2.59 +/− 0.10 | 16.7 +/− 0.8 |
| D29 | 262 | 14.66 +/− 0.47 | 1.42 +/− 0.08 | 1.04 +/− 0.10 | **1.33 +/− 0.15 | **1.92 +/− 0.8 |

2 mg/kg/day

| | | | | | | |
|---|---|---|---|---|---|---|
| D0 | 282 | 6.49 +/− 0.13 | 1.04 +/− 0.02 | 0.88 +/− 0.06 | 2.33 +/− 0.12 | 12.7 +/− 0.6 |
| D21 | 253 | 14.66 +/− 0.73 | 1.49 +/− 0.04 | 1.01 +/− 0.05 | 2.67 +/− 0.17 | 17.0 +/− 1.0 |
| D29 | 261 | 13.70 +/− 0.66 | 1.52 +/− 0.06 | 0.93 +/− 0.04 | **1.38 +/− 0.13 | **21.0 +/− 1.4 |

N = 6 per group, m +/− SEM
**p > 0.01 t test by paired series between D29 and D21.

TABLE II
Effects of minimum active doses of metformin and glibenclamide administered alone or
in combination, on the biological parameters of the diabetic rats. Treatment over eight
days at two doses per day

| | | | | | |
|---|---|---|---|---|---|
| Weight | Glycaemia | Cholesterol | Triglycerides | Lactates | Insulinaemia |
| (g) | mM/l | mM/l | mM/l | mM/l | μU/ml |

Metformin alone 30 mg/kg/day

| | | | | | | |
|---|---|---|---|---|---|---|
| D0 | 288 | 6.38 +/− 0.23 | 1.00 +/− 0.07 | 0.96 +/− 0.12 | 2.47 +/− 0.15 | 12.8 +/− 0.2 |
| D21 | 251 | 14.41 +/− 0.36 | 1.57 +/− 0.08 | 1.05 +/− 0.03 | 2.53 +/− 0.09 | 17.5 +/− 0.8 |
| D29 | 247 | **12.35 +/− 0.49 | 1.48 +/− 0.06 | 0.96 +/− 0.05 | *2.70 +/− 0.11 | 17.3 +/− 0.7 |

Glibenclamide alone 2 mg/kg/day

| | | | | | | |
|---|---|---|---|---|---|---|
| D0 | 282 | 6.49 +/− 0.13 | 1.04 +/− 0.02 | 0.88 +/− 0.06 | 2.33 +/− 0.12 | 12.7 +/− 0.6 |
| D21 | 253 | 14.66 +/− 0.73 | 1.49 +/− 0.04 | 1.01 +/− 0.05 | 2.67 +/− 0.17 | 17.0 +/− 1.0 |
| D29 | 261 | **13.70 +/− 0.66 | 1.52 +/− 0.06 | 0.93 +/− 0.04 | **1.38 +/− 0.13 | **21.0 +/− 1.4 |

Met-Glibenclamide combination

| | | | | | | |
|---|---|---|---|---|---|---|
| D0 | 289 | 6.28 +/− 0.12 | 1.03 +/− 0.05 | 0.99 +/− 0.08 | 2.53 +/− 0.14 | 12.5 +/− 0.8 |
| D21 | 251 | 14.56 +/− 0.30 | 1.55 +/− 0.04 | 1.07 +/− 0.06 | 2.55 +/− 0.09 | 17.3 +/− 1.1 |
| D29 | 254 | **11.42 +/− 0.23 | *1.37 +/− 0.02 | *0.88 +/− 0.04 | **1.29 +/− 0.06 | *20.3 +/− 1.2 |

N = 6 per group, m +/− SEM,
*p > 0.05
**p > 0.01 t test by paired series between D29 and D21.

A synergism of the effects of metformin with glibenclamide is observed on glycaemia. The combination of the two substances significantly decreases the cholesterol and triglycerides, which are increased in the STZ rats. The effects of glibenclamide are found in full on the insulinaemia and on the lactates.

In the latter case, glibenclamide not only decreases the lactates, but also suppresses the effects of metformin, which has a tendency to increase them.

The statistical analysis ran between D29 after the treatment and D21. To specify the type of synergy, additive or potentiating, a Student t test was performed on the percentages of variation of the glycaemia (Table III). From the above results, it is already possible to assert that there is potentiation of the two substances on the levels of cholesterol and triglycerides. Furthermore, the effects of glibenclamide on the insulinaemia are conserved. Finally, glibenclamide suppresses the effects of metformin on lactates.

TABLE III
Study of the decrease in glycaemia between D21 and D29
The results are expressed as a percentage of variation, the values
at D21 being taken as equal to 100%.

| | |
|---|---|
| Metformin 30 mg/kg/day | 86.03 +/− 4.29 |
| Glibenclamide 2 mg/kg/day | 93.49 +/− 1.15 |
| Combination | 78.46 +/− 0.42** |

N = 6 m +/− SEM
**p > 0.01 comparison between combination and glibenclamide ns between combination and metformin

This study shows that there is indeed synergy of the effects of metformin and glibenclamide. The sum of the decreases in glycaemia (13.7% with metformin alone and 6.51% with glibenclamide alone) obtained with the products administered alone (20.21%) is slightly less than that obtained with the combination (21.54%). However, it is difficult to assert that there is potentiation of the effects. An at least additive synergy of the effects of the two products genuinely exists.

### CONCLUSION

The concomitant administration of the two minimum active doses of metformin, 30 mg/kg/day, and of glibenclamide, 2 mg/kg/day, for eight days and at a rate of two administrations per day to NIDD STZ rats results in:

a synergistic effect on the decrease in glycaemia,

a potentiating effect on the decrease in the levels of cholesterol and triglycerides,

an increase in insulinaemia specific to glibenclamide,

a decrease in lactates specific to glibenclamide, with suppression of the effects of metformin on this parameter.

There is thus overall a potentiation of the effects of the two oral antidiabetics, when they are administered in combination at minimum active doses.

1. Novel antidiabetic medicinal products consisting of a combination of an antidiabetic biguanide and an antidiabetic sulfonamide, at low doses, in combination or as a mixture with one or more inert, pharmaceutically acceptable excipients.

2. Novel antidiabetic medicinal products according to claim 1, in which the biguanide is chosen from the group formed by metformin, buformin and phenformin, and also salts with a therapeutically compatible mineral acid or organic acid.

3. Novel antidiabetic medicinal products according to claim 1, in which the sulfonamide corresponds to the general formula: 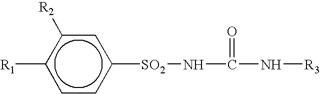
in which R₁ represents an NH₂, a CH₃, a Cl or a group 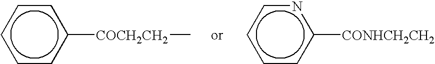
or alternatively R₁ forms with R₂ a group: 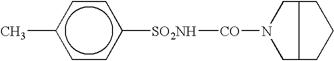
R₂ represents H or NH₂
R₃ represents a 4-n-butyl group, a cyclohexyl group or a group:

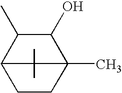

4. Novel antidiabetic medicinal products according to one of claims 1 to 3, in which the sulfonamide is glibenclamide.

5. Novel antidiabetic medicinal products according to one of claims 1 to 3, in which the sulfonamide is gliclazide.

6. Novel antidiabetic medicinal products according to one of claims 1 to 5, in which the dose ratio between the biguanide and the sulfamide ranges from 10:1 to 45:1.

7. Novel antidiabetic medicinal products according to one of claims 1 to 5, in which the weight ratio of active principles ranges from 50:5 mg to 100:2.5 mg.

8. Novel antidiabetic medicinal products according to one of claims 1 to 7, which are in one of the forms suitable for oral administration.

9. Novel antidiabetic medicinal products according to claim 8, in which the inert, non-toxic, solid or liquid pharmaceutical excipient is chosen from mineral fillers and organic fillers.

10. Process for the preparation of pharmaceutical compositions containing a biguanide-sulfonamide combination, characterised in that it is carried out according to the known current processes of pharmacotechnology.
